(19) United States
(12) Patent Application Publication
(10) Pub. No.: US 2021/0079331 A1
(43) Pub. Date: Mar. 18, 2021

(54) BIOLOGICAL DETECTION CARTRIDGE AND METHOD FOR PERFORMING THE SAME

(71) Applicant: Delta Electronics, Inc., Taoyuan City (TW)

(72) Inventors: Chi-Han Chiou, Taoyuan City (TW); Shu-Hsien Liao, Taoyuan City (TW)

(21) Appl. No.: 16/886,414

(22) Filed: May 28, 2020

Related U.S. Application Data
(60) Provisional application No. 62/900,763, filed on Sep. 16, 2019.

(51) Int. Cl. C12M 3/00; C12M 1/12

(52) U.S. Cl. CPC C12M 23/42; C12M 25/04

(57) ABSTRACT

The biological detection cartridge includes a sample chamber having a port, plural culture chambers for incubating the sample therein, a channel system configured to deliver the sample into each of the culture chambers, plural quantitative chambers, and plural concave structures. The channel system includes a curved channel and plural inlet channels, the curved channel is communicated with the sample chamber, and each inlet channel is communicated with the curved channel and a corresponding culture chamber. Each quantitative chamber is disposed between a corresponding inlet channel and a corresponding culture chamber. Each concave structure is disposed between a corresponding quantitative chamber and a corresponding culture chamber. The concave structure includes a first hole disposed close to the culture chamber.

### CROSS-REFERENCE TO RELATED APPLICATION

This application claims the benefit of U.S. Provisional Application No. 62/900,763 filed on Sep. 16, 2019, and entitled “BIOLOGICAL DETECTION CARTRIDGE AND METHOD”, the entirety of which is hereby incorporated by reference.

### FIELD OF THE INVENTION

The present invention relates to a biological detection cartridge and a method for performing the same, and more particularly to a biological detection cartridge and a method for performing the same for an antimicrobial susceptibility test (AST).

### BACKGROUND OF THE INVENTION

The existing standard antimicrobial susceptibility test is performed by using a 96-well plate. FIG. 1 schematically illustrates a 96-well plate for performing an antimicrobial susceptibility test. As shown in FIG. 1, the 96-well plate 1 includes 96 wells W. A process of performing the antimicrobial susceptibility test will be described as follows. Firstly, antimicrobial medicines, such as antibiotics, are added into the wells W. Then, a bacterial solution is added into the wells W containing the antimicrobial medicines. After incubation for 16 to 20 hours, the bacteria growth is observed with naked eyes though the bottom side of the 96-well plate 1. Consequently, the drug resistance of the bacteria can be detected. This method is capable of testing a variety of drug susceptibility and strain determination and observing the test results with naked eyes. Due to these advantages, this method is gold-standard method of the antimicrobial susceptibility test.

However, this method still has some drawbacks. For example, since the antimicrobial medicines are serially diluted to form a concentration gradient, the sample loading procedure is complicated. Moreover, since only one lid is placed over the 96-well plate 1 to cover the openings, a cross-contamination problem is readily generated when the 96-well plate 1 is transported. Moreover, since the volume of the 96-well plate 1 and the volume of the sample drop (e.g., about 100-150 μl) are large, the cost of waste disposal and the risk of contamination increase.

For overcoming the drawbacks of the conventional technologies, there is a need of providing an improved biological detection cartridge and an improved method for performing an antimicrobial susceptibility test while simplifying sample loading and avoiding contamination.

### SUMMARY OF THE INVENTION

An object of the present invention provides an improved biological detection cartridge and a method for performing the same, which can achieve automatic liquid filling, simplify sample loading, and facilitate the antimicrobial susceptibility test.

Another object of the present invention provides an improved biological detection cartridge and a method for performing the same, which can facilitate concentrating the microorganisms at the bottom of the culture chamber for easy observing the incubation results.

An additional object of the present invention provides an improved biological detection cartridge and a method for performing the same, which can effectively achieve quantitative sample loading and avoid sample loading error.

A further object of the present invention provides an improved biological detection cartridge and a method for performing the same, which can prevent the risk of contamination and infection, and provide safety protection and good incubation environment.

In accordance with an aspect of the present invention, a biological detection cartridge is provided. The biological detection cartridge includes a sample chamber having a port, a plurality of culture chambers for incubating the sample therein, a channel system configured to deliver the sample into each of the culture chambers, a plurality of quantitative chambers, and a plurality of concave structures. The channel system includes a curved channel and a plurality of inlet channels, the curved channel is communicated with the sample chamber, and each of the inlet channels is communicated with the curved channel and a corresponding one of the culture chambers. Each of the quantitative chambers is disposed between a corresponding one of the inlet channels and a corresponding one of the culture chambers. Each of the concave structures is disposed between a corresponding one of the quantitative chambers and a corresponding one of the culture chambers. Each of the concave structures includes a first hole disposed close to the culture chamber.

In an embodiment, the curved channel is substantially a continuous S-shaped channel, and each of the inlet channels is connected to a bending portion of the curved channel away from the culture chamber.

In an embodiment, when the biological detection cartridge is placed vertically to have the culture chamber located below the quantitative chamber, a connection point of the inlet channel and the curved channel is located at a relatively high point of the curved channel.

In an embodiment, the concave structure includes a tapered structure at a bottom end of the quantitative chamber, a tapered structure at a top end of the culture chamber, and a neck portion connecting the two tapered structures.

In an embodiment, the first hole is disposed on the tapered structure at the top end of the culture chamber.

In an embodiment, a diameter of the first hole is ranged from 0.1 mm to 1 mm.

In an embodiment, a narrowest width of the concave structure is ranged from 1 mm to 4 mm.

In an embodiment, the plurality of culture chambers contain different amounts of antimicrobial medicines.

In an embodiment, the culture chamber has a circular bottom or a bottom tip.

In an embodiment, the bottom tip has an inclined plane.

In an embodiment, the biological detection cartridge further includes a bottom layer, a channel layer, and a cover layer, wherein at least one of the bottom layer and the cover layer is a hydrophilic film.

In an embodiment, the biological detection cartridge further includes a cartridge body and a cover layer, wherein the cover layer is a hydrophilic film.

In an embodiment, the biological detection cartridge further includes a waste chamber connected to a downstream end of the curved channel, wherein the waste chamber has an exit.

In an embodiment, the biological detection cartridge further includes a second hole disposed on the quantitative chamber.

In an embodiment, the first hole is biased toward one sidewall of each of the concave structures and away from the sample chamber.

In accordance with another aspect of the present invention, a method for performing a biological detection cartridge includes the following steps. First, the biological detection cartridge described above is provided. Then the biological detection cartridge is inclined to have the sample chamber higher than the channel system, and the sample is loaded through the port, so that the sample flows into the curved channel and each of the inlet channels and the quantitative chambers. Subsequently, the biological detection cartridge is vertically placed, so that the sample flows down to the culture chambers.

In an embodiment, before the biological detection cartridge is vertically placed, the openings of the biological detection cartridge are sealed by attaching a film thereon.

The above contents of the present invention will become more readily apparent to those ordinarily skilled in the art after reviewing the following detailed description and accompanying drawings, in which:

### BRIEF DESCRIPTION OF THE DRAWINGS

FIG. 1 schematically illustrates a 96-well plate for performing an antimicrobial susceptibility test;

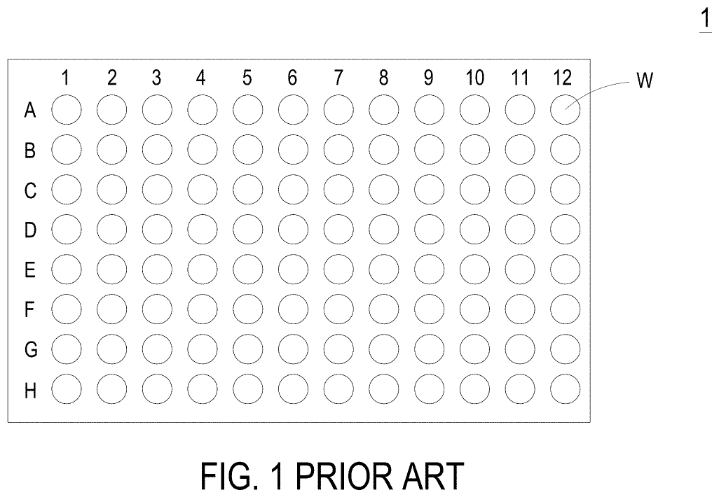

FIG. 2 is a schematic view illustrating a biological detection cartridge according to a first embodiment of the present invention;

FIG. 3 is a schematic exploded view illustrating the biological detection cartridge of FIG. 2;

FIG. 4 schematically illustrates the operation flow of the biological detection cartridge;

FIG. 5 schematically illustrates the biological detection cartridge is inclined;

FIG. 6 is a schematic view illustrating a biological detection cartridge according to a second embodiment of the present invention;

FIG. 7 is a schematic view illustrating a biological detection cartridge according to a third embodiment of the present invention;

FIG. 8 is a schematic view illustrating a biological detection cartridge according to a fourth embodiment of the present invention;

FIG. 9 shows an actual operation of the biological detection cartridge using a red blood cell solution for experiment;

FIG. 10 shows the experimental result of the actual operation of the biological detection cartridge using the bacterial solution;

FIG. 11 is a schematic view illustrating a biological detection cartridge according to a fifth embodiment of the present invention;

FIG. 12 is a schematic exploded view illustrating the biological detection cartridge of the fifth embodiment of the present invention;

FIG. 13 shows a different perspective view of the cartridge body of FIG. 12;

FIG. 14 shows a schematic view illustrating the biological detection cartridge of the fifth embodiment placed on a loading rack;

FIG. 15 shows a schematic view illustrating the biological detection cartridge of the fifth embodiment placed on a culture rack;

FIG. 16 schematically illustrates the operation flow of the biological detection cartridge of the fifth embodiment;

FIG. 17 shows a schematic view illustrating the biological detection cartridge of the fifth embodiment placed on an observation rack; and

FIG. 18 shows an actual operation of the biological detection cartridge of the fifth embodiment.

### DETAILED DESCRIPTION OF THE PREFERRED EMBODIMENT

The present invention will now be described more specifically with reference to the following embodiments. It is to be noted that the following descriptions of preferred embodiments of this invention are presented herein for purpose of illustration and description only. It is not intended to be exhaustive or to be limited to the precise form disclosed.

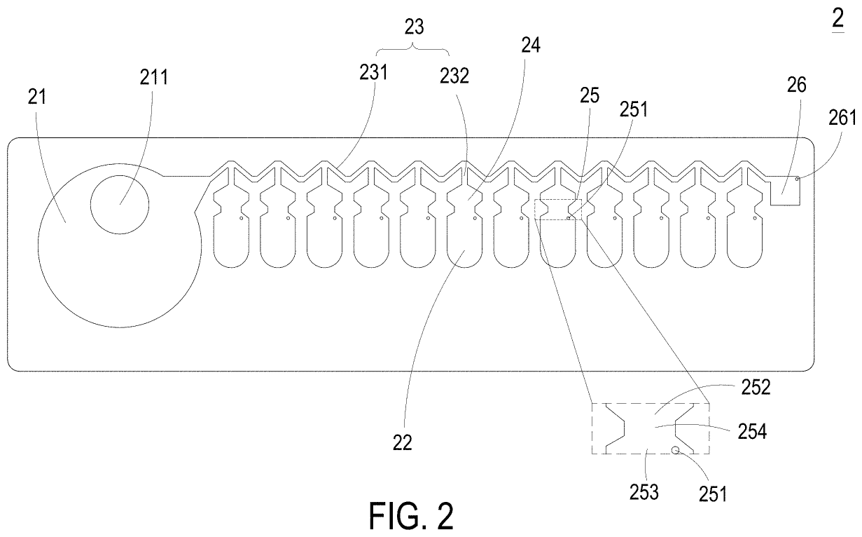

FIG. 2 is a schematic view illustrating a biological detection cartridge according to a first embodiment of the present invention. As shown in FIG. 2, a biological detection cartridge 2 includes a sample chamber 21, a plurality of culture chambers 22, a channel system 23, a plurality of quantitative chambers 24, and a plurality of concave structures 25. The sample chamber 21 includes a port 211 for loading a sample, and the culture chambers 22 are used for incubating the sample therein. The channel system 23 is configured to deliver the sample into each of the culture chambers 22. The channel system 23 includes a curved channel 231 and a plurality of inlet channels 232. The curved channel 231 is communicated with the sample chamber 21, and each of the inlet channels 232 is communicated with the curved channel 231 and a corresponding one of the culture chambers 22, so that the sample is able to flow into each of the culture chambers 22 through the sample chamber 21, the curved channel 231, and the inlet channels 232. Each of the quantitative chambers 24 is disposed between a corresponding one of the inlet channels 232 and a corresponding one of the culture chambers 22. In other words, the two ends of the quantitative chamber 24 are communicated with the inlet channel 232 and the culture chamber 22, respectively. Each of the concave structures 25 is disposed between a corresponding one of the quantitative chambers 24 and a corresponding one of the culture chambers 22. In other words, the two ends of the concave structure 25 are communicated with the quantitative chamber 24 and the culture chamber 22, respectively.

In an embodiment, the concave structure 25 includes a tapered structure 252 at the bottom end of the quantitative chamber 24, a tapered structure 253 at the top end of the culture chamber 22, and a neck portion 254 connecting the two tapered structures 252 and 253. The concave structure 25 includes a first hole 251 disposed close to the culture chamber 22, e.g. disposed on the tapered structure 253 at the top end of the culture chamber 22. The first hole 251 is biased toward one sidewall of the culture chamber 22, e.g. biased toward a right sidewall or a left sidewall of the culture chamber 22 to form an asymmetric structure. In other words, the first hole 251 is located at a right side or a left side of a longitudinal profile passing through the inlet channel 232, so as to form the asymmetric structure. In an embodiment, the first hole 251 is located away from the sample chamber 21. As shown in FIG. 2, the sample chamber 21 is located at the left side of a longitudinal profile passing through the inlet channel 232 and the first hole 251 is located at the right side thereof.

In an embodiment, a diameter of the first hole 251 is ranged from 0.1 mm to 1 mm, and the number of the first hole 251 on each of the concave structure 25 is not limited to one, and may be plural, as long as an asymmetric structure can be formed.

In an embodiment, a width of the neck portion 254 of the concave structure 25 is smaller than a width of the quantitative chamber 24 and a width of the culture chamber 22. The narrowest width of the concave structure 25 is ranged from 1 mm to 4 mm, which is able to prevent the sample from flowing back to the quantitative chamber 24 from the culture chamber 22.

In an embodiment, the curved channel 231 is substantially a continuous S-shaped channel, and each of the inlet channels 232 is connected to a bending portion of the curved channel 231 away from the culture chamber 22. Therefore, when the biological detection cartridge 2 is placed vertically to have the culture chamber 22 located below the quantitative chamber 24, the connection point of the inlet channel 232 and the curved channel 231 is located at a relatively high point of the curved channel 231, and the inlet channel 232 runs vertically.

In an embodiment, the sample is a biological sample containing the microorganisms to be tested. The plurality of culture chambers 22 contain different amounts of antimicrobial medicines in advance for antimicrobial susceptibility test (AST). After fixed amount of the biological sample is added, the plurality of culture chambers 22 contain different concentrations of antimicrobial medicines, and thus the growth of microorganisms under different concentrations of antimicrobial medicines can be observed, and the degree of drug resistance of the microorganisms can be determined.

In an embodiment, the culture chamber 22 has a circular bottom at the side away from the quantitative chamber 24. When the biological detection cartridge 2 is placed vertically for incubation, the circular bottom design facilitates concentrating the microorganisms at the bottom of the culture chamber 22 for easy observation by the operator.

In an embodiment, a downstream end of the curved channel 231 is connected to a waste chamber 26 used to collect excess sample, and the waste chamber 26 has an exit 261 to facilitate exhausting air.

In an embodiment, the biological detection cartridge 2 is made of a transparent material, so as to facilitate observing the liquid flow in the cartridge and the growth of microorganisms in the culture chambers 22.

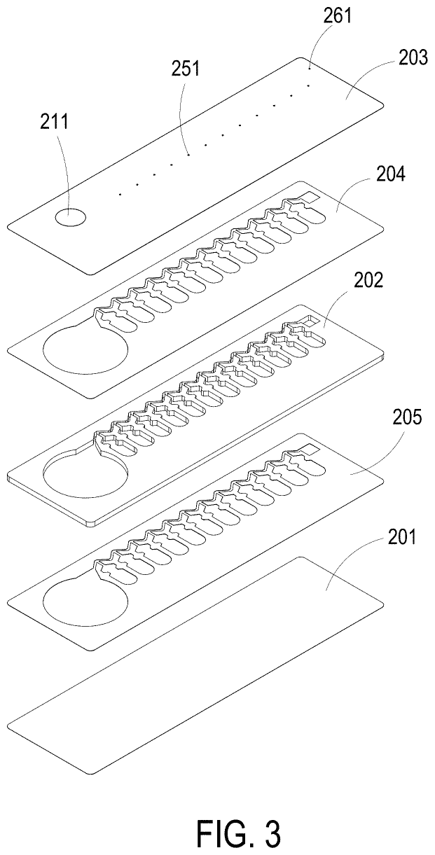

FIG. 3 is a schematic exploded view illustrating the biological detection cartridge of FIG. 2. As shown in FIG. 3, the biological detection cartridge 2 includes a bottom layer 201, a channel layer 202, and a cover layer 203. The channel layer 202 is provided with structures of the channels and the chambers. The cover layer 203 is provided with the port 211, the first hole 251, and the exit 261. The bottom layer 201 is the bottom support of the cartridge and the region where the antimicrobial medicines are dried. The cover layer 203 and the bottom layer 201 covers the upper and lower surfaces of the channel layer 202, respectively, and the cover layer 203, the bottom layer 201, and the channel layer 202 collectively defines the channels and the chambers inside the biological detection cartridge 2. Preferably, at least one of the bottom layer 201 and the cover layer 203 is a hydrophilic film to reduce the flow resistance of the channels, so that the fluid can flow smoothly in the channels.

In an embodiment, the cover layer 203 can be adhered to the upper surface of the channel layer 202 through a first adhesive layer 204, and the first adhesive layer 204 has openings corresponding to the structures of the channels and the chambers of the channel layer 202. Similarly, the bottom layer 201 can be adhered to the lower surface of the channel layer 202 through a second adhesive layer 205, and the second adhesive layer 205 has openings corresponding to the structures of the channels and the chambers of the channel layer 202. For example, the first adhesive layer 204 and the second adhesive layer 205 may be double-sided tapes, or glue directly coated between two structural layers, but not limited thereto. Certainly, the cover layer 203 and the bottom layer 201 can also be bonded to the channel layer 202 by ultrasonic welding without providing an adhesive layer therebetween. Alternatively, one of the cover layer 203 and the bottom layer 201 may be integrally formed with the channel layer 202, and the other layer may be bonded to the channel layer 202 by the adhesive layer or ultrasonic welding.

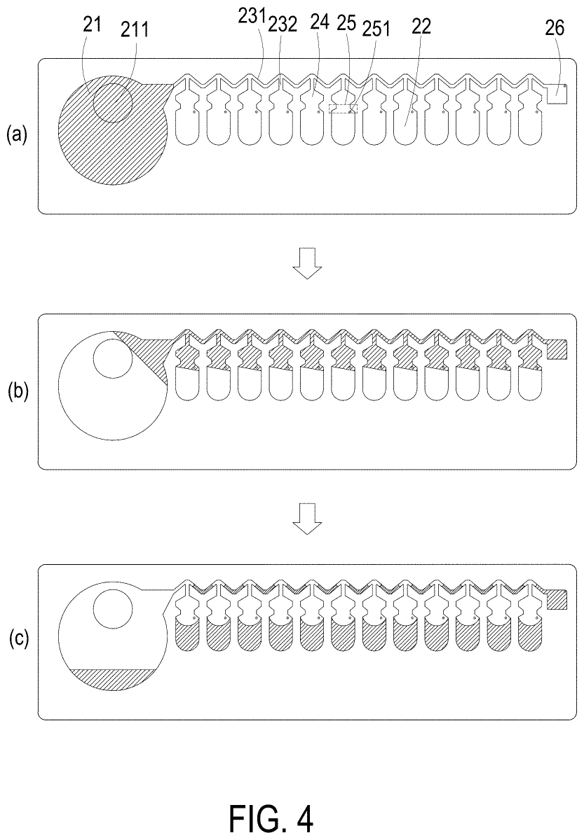

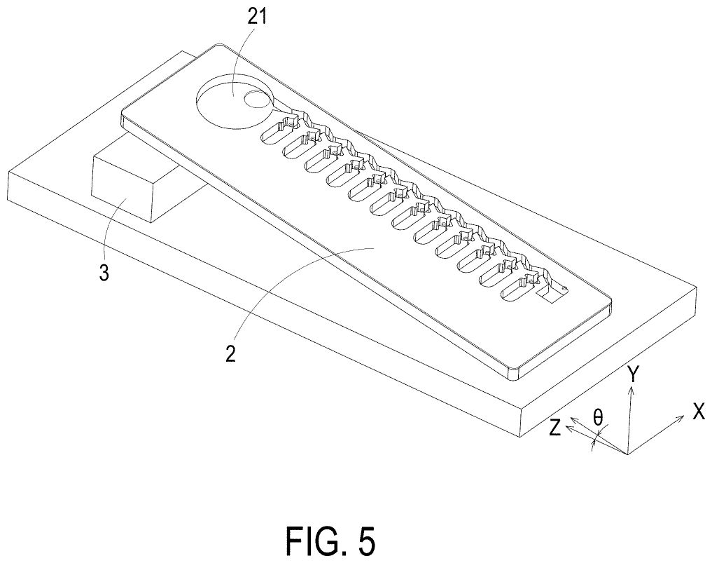

FIG. 4 schematically illustrates the operation flow of the biological detection cartridge, and FIG. 5 schematically illustrates the biological detection cartridge is inclined. First, before adding the sample, the biological detection cartridge 2 (referred as the cartridge 2) is inclined to have the sample chamber 21 higher than the channel system 23. For example, the cartridge 2 is placed on a tilting jig 3 (as shown in FIG. 5) to lift up the cartridge 2 on the side of the sample chamber 21. The tilt angle θ of the cartridge 2 is greater than 10°, for example, between 10° and 80°, but not limited thereto. Then, as shown in step (a) of FIG. 4, the sample is added into the sample chamber 21 through the port 211. Subsequently, as shown in step (b) of FIG. 4, the sample flows into the curved channel 231 and each of the inlet channels 232, the quantitative chambers 24 and the concave structures 25 from the sample chamber 21 due to gravity and capillary force, and stops at the positions of the first holes 251 of the concave structures 25. Since the first hole 251 is biased toward one sidewall of the culture chamber 22, for example, the right sidewall shown in FIG. 4, when the sample stops at the first hole 251, the leading edge of the sample is asymmetric.

Afterwards, the cartridge 2 is removed from the tilting jig 3 and then placed vertically, i.e., along Y axis shown in FIG. 5, so that the culture chamber 22 is located below the quantitative chamber 24, and the connection point of the inlet channel 232 and the curved channel 231 is located at a relatively high point of the curved channel 231. Meanwhile, the sample in the inlet channel 232 and the quantitative chamber 24 flows down to the culture chamber 22 because of the left-right imbalance and the gravity of the liquid. Accordingly, the liquid in the inlet channel 232 and the quantitative chamber 24 is fully evacuated, and thus, the liquid in the culture chamber 22 is disconnected with the liquid remaining in the curved channel 231. Moreover, due to the gravity, the liquid in the curved channel 231 remains in the bending portions at the relatively low points of the curved channel 231 to further separate each culture chamber 22, thereby avoiding cross contamination, as shown in step (c) of FIG. 4. In other words, the curved channel 231 and the inlet channels 232 of the present invention work collaboratively to have an intercepting function, which can separate each culture chamber 22 to prevent pollution and infection risks and provide safety protection. In addition, since the liquid flowing into the quantitative chamber 24 first stops at the position of the first hole 251 and then flows down to the culture chamber 22, the amount of the liquid flowing into the culture chamber 22 can be further quantified.

In an embodiment, after the sample is added, all the openings of the cartridge 2 can be sealed by attaching a film or a lid on the top of the cartridge 2 to prevent the sample from volatilizing during incubation. Finally, the cartridge 2 is placed vertically for microorganism incubation. After incubation for a period of time, e.g. 16 to 20 hours, the cartridge 2 is placed on an observation rack to observe the incubation results with naked eyes. Meanwhile, the sample is trapped in the culture chamber 22 due to the anti-backflow design of the concave structure 25.

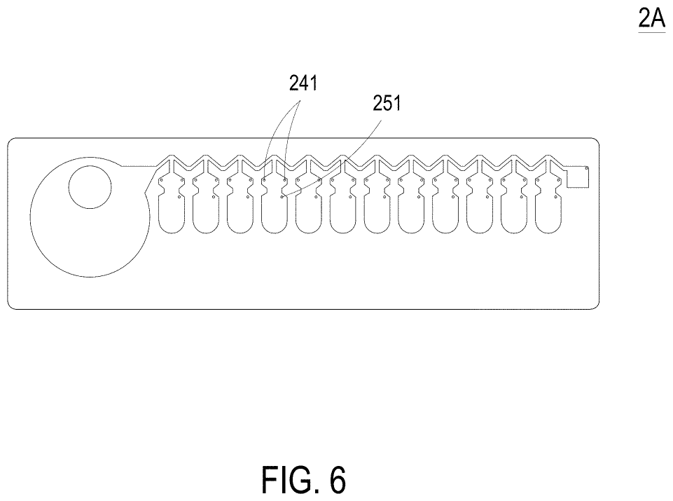

FIG. 6 is a schematic view illustrating a biological detection cartridge according to a second embodiment of the present invention. Compared to the biological detection cartridge 2 shown in FIG. 2, in addition to the first hole 251, the biological detection cartridge 2A shown in FIG. 6 further includes one or more second holes 241. The second holes 241 may be disposed between the inlet channel 232 and the concave structure 25. That is to say, the second hole 241 is disposed on the quantitative chamber 24, and is also opened in the cover layer 203. For example, the quantitative chamber 24 has two second holes 241 disposed symmetrically on a side close to the inlet channel 232, but not limited thereto.

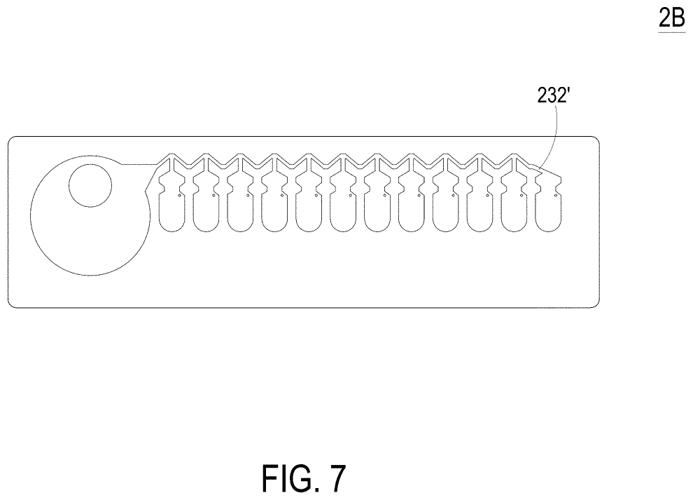

FIG. 7 is a schematic view illustrating a biological detection cartridge according to a third embodiment of the present invention. Compared to the biological detection cartridge 2 shown in FIG. 2, the biological detection cartridge 2B shown in FIG. 7 does not include a waste chamber. To enable the liquid to flow into the last quantitative chamber 24 smoothly, the last inlet channel 232′ is connected to the relatively low point of the curved channel 231 and flows diagonally into the last quantitative chamber 24.

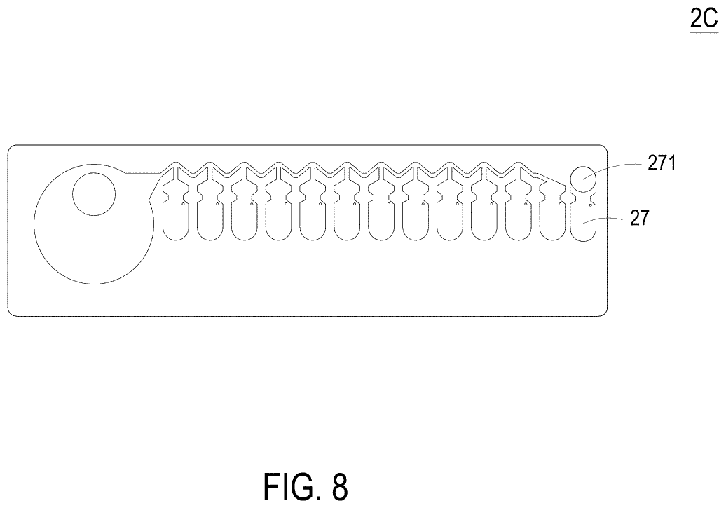

FIG. 8 is a schematic view illustrating a biological detection cartridge according to a fourth embodiment of the present invention. Compared to the biological detection cartridge 2B shown in FIG. 7, the biological detection cartridge 2C shown in FIG. 8 further includes a negative control culture chamber 27 only for loading an incubation solution instead of a biological sample containing the microorganisms, so as to be used as a control group for microorganism incubation. The negative control culture chamber 27 has its own port 271 for adding the incubation solution.

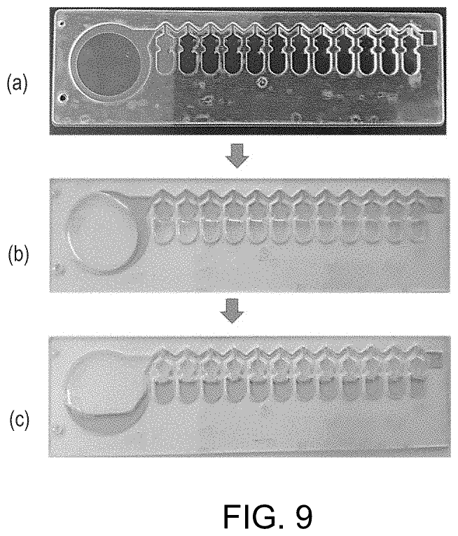

FIG. 9 shows an actual operation of the biological detection cartridge using a red blood cell solution for experiment, wherein the hematocrit (HCT) of the red blood cell solution is 4%. First, the cartridge was placed on the tilting jig (step (a)), and then 1500 μL of the red blood cell solution was added through the port. Subsequently, the liquid automatically flowed into each of the quantitative chambers and the concave structures and stopped at the positions of the first holes (step (b)). After that, the cartridge was placed vertically, and meanwhile, the liquid in each of the inlet channels and the quantitative chambers flowed down to the culture chambers (step (c)). By the colored red blood cell solution, the liquid flow in the cartridge can be clearly seen, and from this simulation experiment, it is demonstrated that the biological detection cartridge of the present invention has the advantages of convenient sample loading, quantification, and observation.

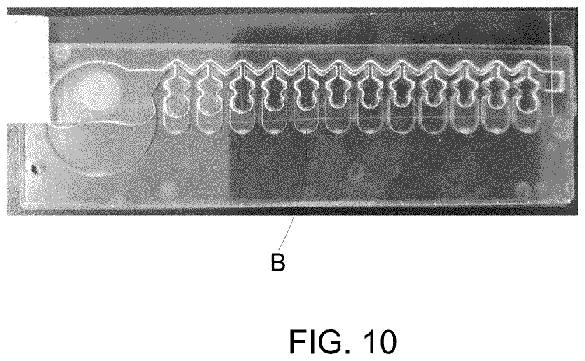

FIG. 10 shows the experimental result of the actual operation of the biological detection cartridge using the bacterial solution. 1500 μL of the bacterial solution was added through the port, and after the bacterial solution was automatically filled into each of the culture chambers, the incubation was performed at 36° C. for 20 hours. Then the bacterial pellets B were observed at the bottom of the culture chambers.

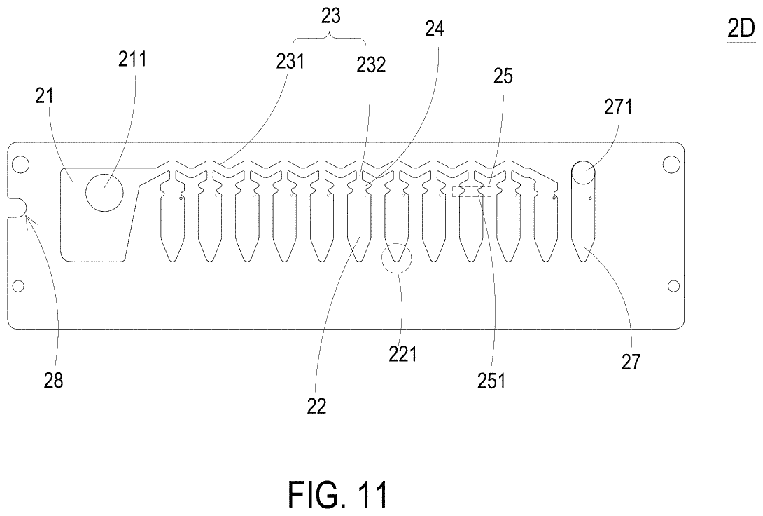

FIG. 11 is a schematic view illustrating a biological detection cartridge according to a fifth embodiment of the present invention. In this embodiment, the configurations of the sample chamber 21, the culture chamber 22, the channel system 23, the quantitative chamber 24, the concave structure 25, and the negative control culture chamber 27 of the biological detection cartridge 2D are substantially the same as those of the biological detection cartridge 2C in the fourth embodiment shown in FIG. 8, and the major difference therebetween is the structural design at the bottom of the culture chamber 22. In the foregoing first to fourth embodiments, the culture chamber 22 has a circular bottom, while in this embodiment, the bottom of the culture chamber 22 is provided with a significantly tapered tip 221, which greatly facilitates concentrating the microorganisms at the tapered tip 221 of the culture chamber 22 for much easier observation by the operator.

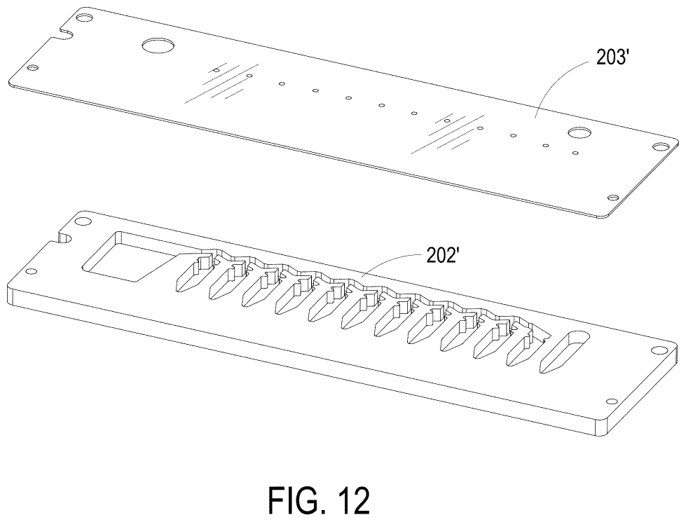

FIG. 12 is a schematic exploded view illustrating the biological detection cartridge of the fifth embodiment of the present invention. Different from the biological detection cartridge 2 including the bottom layer 201, the channel layer 202, and the cover layer 203 shown in FIG. 3, the biological detection cartridge 2D of the fifth embodiment includes a cartridge body 202′ and a cover layer 203′. In other words, the bottom layer and the channel layer are integrally formed as the cartridge body 202′, and thus the biological detection cartridge 2D of the fifth embodiment does not include an independent bottom layer. In this embodiment, the cover layer 203′ is a hydrophilic film to reduce the flow resistance of the channels, so that the fluid can flow smoothly in the channels. The hydrophilic film may include an adhesive layer to facilitate adhesion to the cartridge body 202′. In addition, the cover layer 203′ is preferably a transparent layer to facilitate operation and observation during incubation.

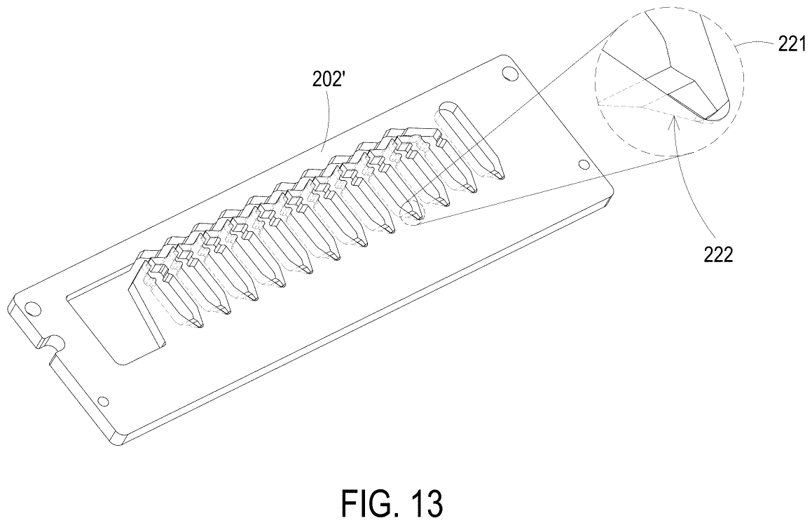

FIG. 13 shows a different perspective view of the cartridge body of FIG. 12, and the internal structures of the chambers are shown with dotted lines. As shown in FIG. 13, there is an inclined plane 222 at the tip 221 of the culture chamber 22, which is substantially inclined from the bottom surface to the top surface of the cartridge body 202′. When the biological detection cartridge 2D is placed vertically for incubation, the inclined plane 222 facilitates the sample and the microorganisms sliding along the inclined plane 222 and gathering to the tip end at the bottom of the culture chamber 22 to facilitate subsequent incubation and observation. In some variations, the inclined plane 222 may be a continuous slope, a multi-stage slope, or a combination of a slope and a curved surface, but is not limited thereto.

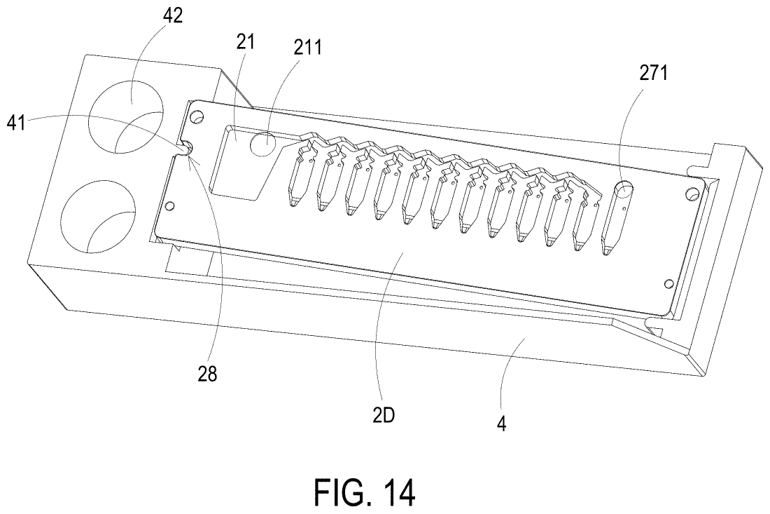

FIG. 14 shows a schematic view illustrating the biological detection cartridge of the fifth embodiment placed on a loading rack. As shown in FIG. 14, the biological detection cartridge 2D of this embodiment further has a foolproof design, which facilitates correctly placing the biological detection cartridge 2D on the loading rack 4 for sample loading. Particularly, the biological detection cartridge 2D and the loading rack 4 have corresponding alignment or engaging structures. For example, the biological detection cartridge 2D has a recess 28, and the loading rack 4 has a corresponding protrusion 41. When the sample is to be loaded, the biological detection cartridge 2D can be correctly placed on the loading rack 4 by simply aligning the recess 28 of the biological detection cartridge 2D with the protrusion 41 of the loading rack 4. Since the side of the sample chamber 21 is lifted up, after the sample is added into the sample chamber 21 through the port 211, the sample flows into the curved channel 231 and each of the inlet channels 232, the quantitative chambers 24 and the concave structures 25 from the sample chamber 21 due to gravity and capillary force, and stops at the positions of the first holes 251 of the concave structures 25. Afterwards, all the openings of the cartridge can be sealed by attaching a film on the top of the cartridge to prevent the sample from volatilizing during incubation.

Certainly, the foolproof structures are not limited to the aforementioned recess 28 and protrusion 41, and other structural designs having the foolproof effect can also be applied to the present invention. Further, the loading rack 4 may be provided with a receiving chamber 42 for accommodating a sample bottle, which makes the sample loading more convenient.

On the other hand, the designs of the tip 221 and the inclined plane 222 at the bottom of the culture chamber 22 and the foolproof design for the biological detection cartridge 2D of the fifth embodiment can also be applied to the cartridges of the first to fourth embodiments.

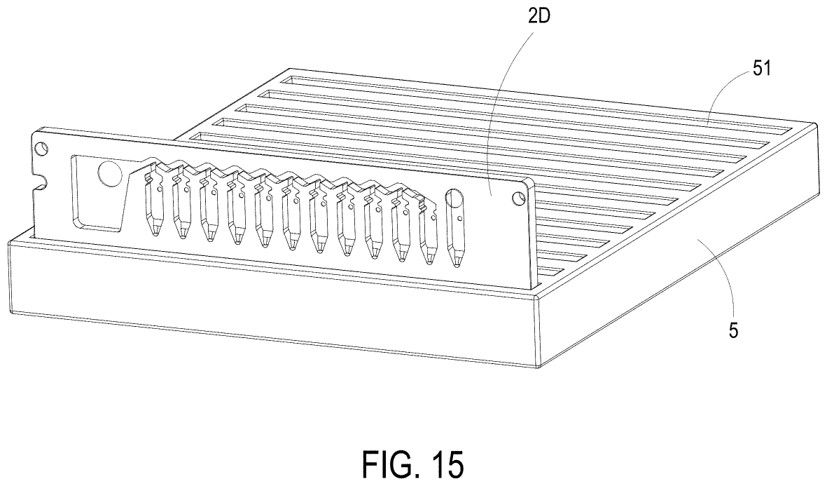

FIG. 15 shows a schematic view illustrating the biological detection cartridge of the fifth embodiment placed on a culture rack. After sample loading and cartridge sealing, the cartridge 2D is removed from the loading rack 4 and then placed vertically in the culture rack 5 for incubation. When the cartridge 2D is placed vertically to have the culture chamber 22 located below the quantitative chamber 24, due to the left-right imbalance and the gravity of the liquid, the sample in the inlet channel 232 and the quantitative chamber 24 flows down to the culture chamber 22, and the liquid in each culture chamber 22 is separated from each other, so cross-contamination during incubation can be avoided. Moreover, the culture rack 5 is provided with a plurality of slots 51 for vertically inserting a plurality of cartridges 2D therein, which facilitates multiple incubations at the same time, such as for multiple incubations of different samples or different antimicrobial medicines.

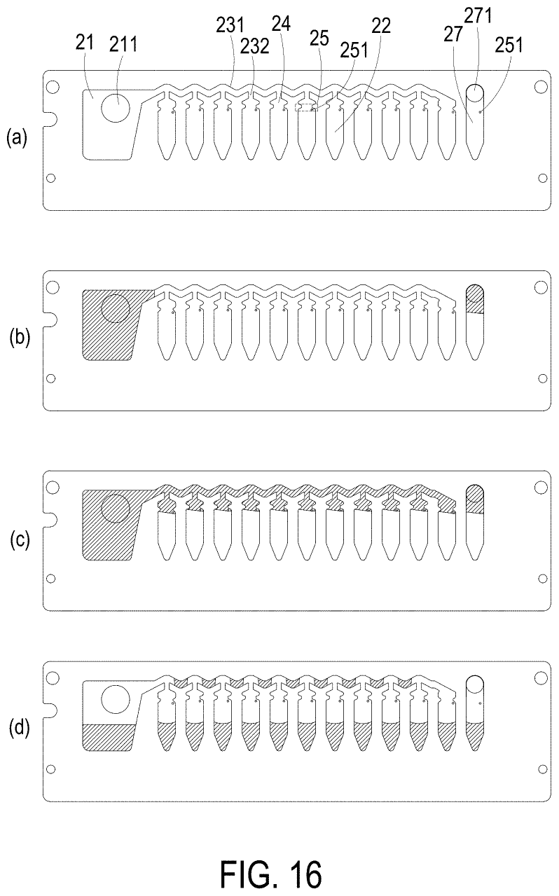

FIG. 16 schematically illustrates the operation flow of the biological detection cartridge of the fifth embodiment. First, before loading the sample, the cartridge 2D is inclined to have the sample chamber 21 higher than the channel system 23. For example, as shown in FIG. 14 and step (a) of FIG. 16, the cartridge 2D is placed on the loading rack 4 to lift up the cartridge 2D on the side of the sample chamber 21. Then as shown in step (b) of FIG. 16, the sample is added to the sample chamber 21 through the left port 211, and the incubation solution is added to the negative control culture chamber 27 through the right port 271. Then as shown in step (c) of FIG. 16, the sample added through the left port 211 flows into the curved channel 231 and each of the inlet channels 232, the quantitative chambers 24 and the concave structures 25 from the sample chamber 21 due to gravity and capillary force, and stops at the positions of the first holes 251 of the concave structures 25, and the leading edge of the sample is asymmetric. Similarly, the incubation solution added through the right port 271 stops at the position of the first hole 251, and the leading edge of the incubation solution is asymmetric. Afterwards, the cartridge 2D is sealed with a film and removed from the loading rack 4. Then the cartridge 2D is placed vertically in the slot 51 of the culture rack 5, and the sample flows down to the culture chamber 22 for incubation, as shown in step (d) of FIG. 16.

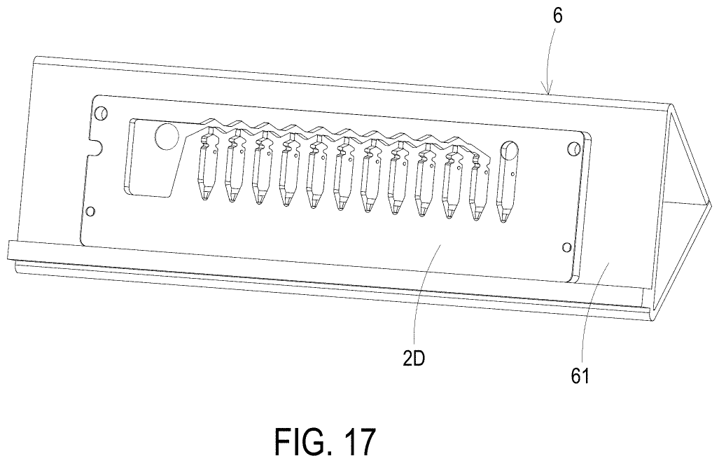

After incubation for a period of time, for example, after about 16 to 20 hours, the incubation results are observed. The incubation results can be directly observed while the cartridge 2D is placed on the culture rack 5. Alternatively, in order to facilitate observation, the present invention also provides a design of an observation rack. FIG. 17 shows a schematic view illustrating the biological detection cartridge of the fifth embodiment placed on an observation rack. As shown in FIG. 17, the observation rack 6 has an inclined surface 61, so that the cartridge 2D can be placed on the observation rack 6 with the bottom surface attached to the inclined surface 61 of the observation rack 6. According to different observation targets, the color of the inclined surface 61 of the observation rack 6 can also be adjusted to facilitate observation. For example, when observing the bacterial pellets, the observation rack 6 can provide a black background to make the slightly white pellets more visible. If a color change of an indicator is to be observed, the observation rack 6 can provide a white background to make the color change more obvious. For instance, adjusting the background color of the observation rack 6 can be achieved by placing colored papers or sheets with different colors on the inclined surface 61, or by forming the observation rack 6 with different colors of plastic, but not limited thereto.

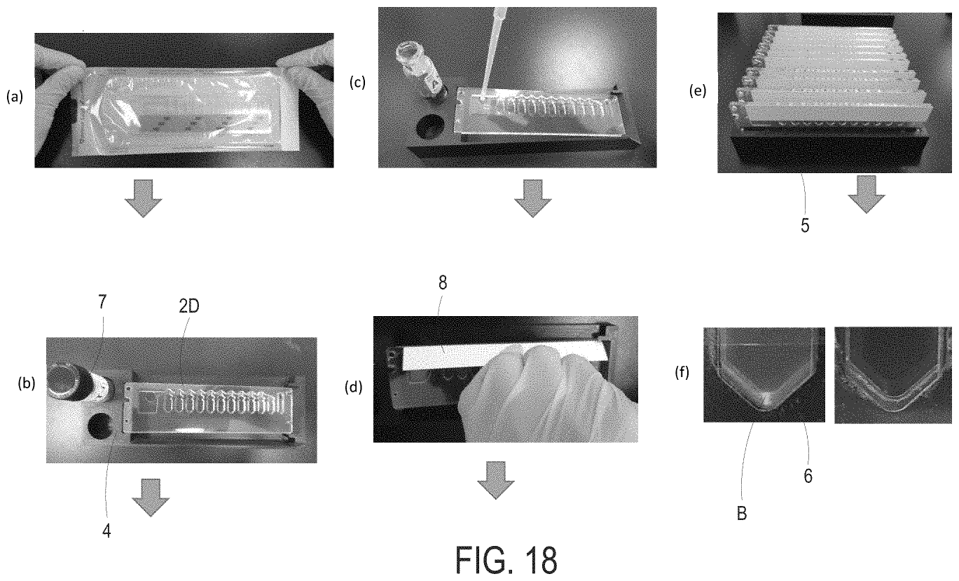

FIG. 18 shows an actual operation of the biological detection cartridge of the fifth embodiment. First, as shown in step (a), the sterilized package was opened to take out the cartridge 2D, and then as shown in step (b), the cartridge 2D and the sample bottle 7 were placed on the loading rack 4. Subsequently, as shown in step (c), 1.5 mL of the sample was dropped through the left port, and 0.1 mL of the incubation solution was dropped through the right port. Then as shown in step (d), a sealing film 8 was attached on the cartridge 2D to seal the openings. Thereafter, as shown in step (e), the cartridge 2D was placed vertically by inserting into the slot of the culture rack 5, so that the sample flows down to the culture chamber for microorganism incubation. After incubation for about 16 to 20 hours, the cartridge 2D was placed on the observation rack 6 to observe the growth of microorganisms. For example, the left figure of step (f) shows the bacterial pellets B at the bottom tip of the culture chamber, and the right figure shows no bacteria in the negative control group.

Therefore, the present invention also provides a method for performing a biological detection cartridge. First, the biological detection cartridge according to any embodiment described above is provided. Then the biological detection cartridge is inclined to have the sample chamber higher than the channel system, for example by placing the biological detection cartridge on the loading rack to lift up the biological detection cartridge on the side of the sample chamber. Afterwards, the sample is loaded through the port, so that the sample flows into the curved channel and each of the inlet channels, the quantitative chambers and the concave structures 25, and stops at positions of the first holes of the concave structures. Subsequently, the openings of the biological detection cartridge are sealed by attaching a film thereon, and then the biological detection cartridge is inserted into the slot of the culture rack to vertically place the biological detection cartridge, so that the sample flows down to the culture chambers. After incubation for a period of time, the biological detection cartridge is placed on the observation rack to observe the incubation results.

From the above descriptions, by the designs of the channels and the holes of the biological detection cartridge in the present invention, the sample can be automatically filled into multiple culture chambers after the sample is loaded through the port. In addition, the culture chamber has the circular bottom or the bottom tip, which facilitates concentrating the microorganisms at the bottom of the culture chamber for easy observation by the operator. Further, the biological detection cartridge includes the quantitative chamber, and with the design of the first hole, the liquid flowing into the quantitative chamber first stops at the position of the first hole and then flows down to the culture chamber, so the amount of the liquid flowing into the culture chamber can be further quantified. Moreover, the biological detection cartridge includes the curved channel and the inlet channels, which can completely intercept and separate each culture chamber through liquid gravity after the cartridge is placed vertically, so as to prevent the risk of contamination and infection, and provide safety protection and good incubation environment. Furthermore, the biological detection cartridge includes a plurality of culture chambers which contain different amounts of antimicrobial medicines in advance, so the cartridge can be further applied for antimicrobial susceptibility test.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A biological detection cartridge, comprising:
a sample chamber having a port for loading a sample;
a plurality of culture chambers for incubating the sample therein;
a channel system configured to deliver the sample into each of the culture chambers and comprising a curved channel and a plurality of inlet channels, wherein the curved channel is communicated with the sample chamber, and each of the inlet channels is communicated with the curved channel and a corresponding one of the culture chambers;
a plurality of quantitative chambers, each of which being disposed between a corresponding one of the inlet channels and a corresponding one of the culture chambers; and
a plurality of concave structures, each of which being disposed between a corresponding one of the quantitative chambers and a corresponding one of the culture chambers,
wherein each of the concave structures comprises a first hole disposed close to the culture chamber.

2. The biological detection cartridge according to claim 1, wherein the curved channel is substantially a continuous S-shaped channel, and each of the inlet channels is connected to a bending portion of the curved channel away from the culture chamber.

3. The biological detection cartridge according to claim 1, wherein when the biological detection cartridge is placed vertically to have the culture chamber located below the quantitative chamber, a connection point of the inlet channel and the curved channel is located at a relatively high point of the curved channel.

4. The biological detection cartridge according to claim 1, wherein the concave structure comprises a tapered structure at a bottom end of the quantitative chamber, a tapered structure at a top end of the culture chamber, and a neck portion connecting the two tapered structures.

5. The biological detection cartridge according to claim 4, wherein the first hole is disposed on the tapered structure at the top end of the culture chamber.

6. The biological detection cartridge according to claim 1, wherein a diameter of the first hole is ranged from 0.1 mm to 1 mm.

7. The biological detection cartridge according to claim 1, wherein a narrowest width of the concave structure is ranged from 1 mm to 4 mm.

8. The biological detection cartridge according to claim 1, wherein the plurality of culture chambers contain different amounts of antimicrobial medicines.

9. The biological detection cartridge according to claim 1, wherein the culture chamber has a circular bottom or a bottom tip.

10. The biological detection cartridge according to claim 9, wherein the bottom tip has an inclined plane.

11. The biological detection cartridge according to claim 1, further comprising a bottom layer, a channel layer, and a cover layer, wherein at least one of the bottom layer and the cover layer is a hydrophilic film.

12. The biological detection cartridge according to claim 1, further comprising a cartridge body and a cover layer, wherein the cover layer is a hydrophilic film.

13. The biological detection cartridge according to claim 1, further comprising a waste chamber connected to a downstream end of the curved channel, wherein the waste chamber has an exit.

14. The biological detection cartridge according to claim 1, further comprising a second hole disposed on the quantitative chamber.

15. The biological detection cartridge according to claim 1, wherein the first hole is biased toward one sidewall of each of the concave structures and away from the sample chamber.

16. A method for performing a biological detection cartridge, comprising steps of:
(a) providing the biological detection cartridge, wherein the biological detection cartridge comprises a sample chamber having a port, a plurality of culture chambers for incubating a sample therein, a channel system configured to deliver the sample into each of the culture chambers, a plurality of quantitative chambers, and a plurality of concave structures, wherein the channel system comprises a curved channel and a plurality of inlet channels, the curved channel is communicated with the sample chamber, and each of the inlet channels is communicated with the curved channel and a corresponding one of the culture chambers, wherein each of the quantitative chambers is disposed between a corresponding one of the inlet channels and a corresponding one of the culture chambers, wherein each of the concave structures is disposed between a corresponding one of the quantitative chambers and a corresponding one of the culture chambers, wherein the concave structure comprises a first hole disposed close to the culture chamber;
(b) inclining the biological detection cartridge to have the sample chamber higher than the channel system, and loading the sample through the port, so that the sample flows into the curved channel and each of the inlet channels and the quantitative chambers; and
(c) vertically placing the biological detection cartridge, so that the sample flows down to the culture chambers.

17. The method according to claim 16, wherein in step (a), the plurality of culture chambers contain different amounts of antimicrobial medicines.

18. The method according to claim 16, wherein in step (b), the biological detection cartridge is placed on a loading rack, and the sample flows into the concave structures and stops at positions of the first holes of the concave structures.

19. The method according to claim 16, wherein in step (c), the biological detection cartridge is inserted into a slot of a culture rack.

20. The method according to claim 16, further comprising a step of attaching a film on the biological detection cartridge to seal openings of the biological detection cartridge.
